(19) United States
(12) Patent Application Publication
(10) Pub. No.: US 2023/0270803 A1
(43) Pub. Date: Aug. 31, 2023

(54) MEDICAL APPARATUS WITH CANNABIS SATIVA L.

(71) Applicant: Pure GREEN PHARMACEUTICALS. INC., West Bloomfield, MI (US)

(72) Inventors: Stephen Goldner, West Bloomfield, MI (US); Debra Kimless, Chadds Ford, PA (US); Ara Kirakosyan, Saline, MI (US)

(21) Appl. No.: 17/637,020

(22) PCT Filed: Aug. 21, 2020
(86) PCT No.: PCT/US2020/047354

§ 371 Date: Feb. 21, 2022

Related U.S. Application Data
(60) Provisional application No. 62/889,648, filed on Aug. 21, 2019.

(51) Int. Cl. A61K 36/185; A61K 9/00; A61K 9/70

(52) U.S. Cl. CPC A61K 36/185; A61K 9/0024; A61K 9/70; A61K 2236/30

(57) ABSTRACT

An example composite material for a medical apparatus includes a matrix material, and a cannabis phase disposed in the matrix material. The cannabis phase includes at least one active chemical. A medical apparatus and a method of making a medical apparatus are also disclosed.

### CROSS REFERENCE TO RELATED APPLICATIONS

This application claims priority to U.S. Provisional Pat. Serial No. 62/889,648, filed Aug. 21, 2019, which is hereby incorporated herein by reference in its entirety.

### BACKGROUND

Plant matter, such as cannabis sativa L., contains active chemicals that are useful for medicinal purposes to treat disease or alleviate symptoms. For instance, the active chemicals, which include (among other things) cannabinoids and terpenes, have been found to exhibit anti-inflammatory, anti-bacterial, anti-fungal, antiemetic, orexigenic, muscle relaxant properties, and other desirable properties.

### SUMMARY

An example composite material for a medical apparatus according to the present disclosure includes, among other possible things, a matrix material, and a cannabis phase disposed in the matrix material, wherein the cannabis phase includes at least one active chemical.

In a further example of the foregoing, the cannabis phase includes at least one of terpenes and flavonoids.

In a further example of any of the foregoing, the cannabis phase includes fibers from a cannabis plant.

In a further example of any of the foregoing, the fibers are bast fibers, hurd fibers, or combinations thereof.

In a further example of any of the foregoing, the matrix is a polymeric material.

In a further example of any of the foregoing, the matrix is a ceramic material.

In a further example of any of the foregoing, the cannabis phase is ground or cut fibers of a cannabis plant.

In a further example of any of the foregoing, the cannabis phase includes at least one cannabis extract.

In a further example of any of the foregoing, the composite is formed into a medical apparatus configured to be implanted into a patient.

In a further example of any of the foregoing, the composite is formed into a tool configured to be used during a medical procedure.

In a further example of any of the foregoing, the cannabis phase includes material from Cannabis sativa L .

A medical apparatus according to an example of the present disclosure includes, among other possible things, a cannabis phase, the cannabis phase including at least one of hurd fibers and bast fibers of a cannabis plant, wherein the medical apparatus intrinsically has at least one of anti-inflammatory, anti-bacterial, anti-fungal, antiemetic, orexigenic, and muscle relaxant properties due to the cannabis phase.

In a further example of any of the foregoing, the medical apparatus is configured to be implanted in a patient.

In a further example of any of the foregoing, the medical apparatus is a tool configured to be used in a medical procedure.

In a further example of any of the foregoing, the medical apparatus is a fabric.

In a further example of any of the foregoing, the cannabis phase includes material from Cannabis sativa L .

A method of making a medical apparatus according to an example of the present disclosure includes, among other possible things, forming fibers from a cannabis plant into a medical apparatus, whereby the medical apparatus intrinsically has at least one of anti-inflammatory, anti-bacterial, anti-fungal, antiemetic, orexigenic, and muscle relaxant properties due to the fibers.

In a further example of any of the foregoing, the method further includes disposing the fibers into a matrix phase.

In a further example of any of the foregoing, the method further includes weaving, knitting, or braiding the fibers.

In a further example of any of the foregoing, the medical apparatus is implantable into a patient.

### BRIEF DESCRIPTION OF THE DRAWINGS

FIGS. 1A-B schematically show a cannabis plant.

FIG. 2 schematically shows an example medical apparatus comprising an example composite material according to the present disclosure.

FIG. 3 schematically shows another example medical apparatus comprising an example composite material according to the present disclosure.

### DETAILED DESCRIPTION

Cannabis sativa L. is a plant that naturally produces various active chemicals that have medicinally useful properties. These chemicals includes cannabinoids, such as cannabidiol (commonly known as CBD) and tetrahydrocannabinol (commonly known as THC). Certain cannabinoids have been shown to exhibit anti-inflammatory, anti-bacterial, anti-fungal, antiemetic, orexigenic, muscle relaxant, or other medicinally beneficial properties.

The present disclosure applies to male, female, or hermaphrodite Cannabis sativa L. plants. It should be understood that the present disclosure is also applicable to other plants in the Cannabaceae family. Furthermore, it should be understood that the present disclosure also applies to synthetic versions of active chemicals from Cannabis sativa L. or other plants from the Cannabaceae family, or synthetic chemicals that mimic the effects of such active chemicals. For simplicity, “cannabis” as used herein includes Cannabis sativa L., other plants from the Cannabaceae family, the aforementioned synthetic chemicals, or combinations thereof.

Other than cannabinoids, cannabis includes other compounds. Terpenes, which are aromatic unsaturated hydrocarbons, are another class of chemicals that are naturally occurring in cannabis. Example terpenes found in Cannabis sativa L. include myrcene, β-caryophyllene, humulene, linalool, limonene and α-pinene. Cannabis sativa L. also includes flavonoids, such as cannflavin A and cannflavin B.

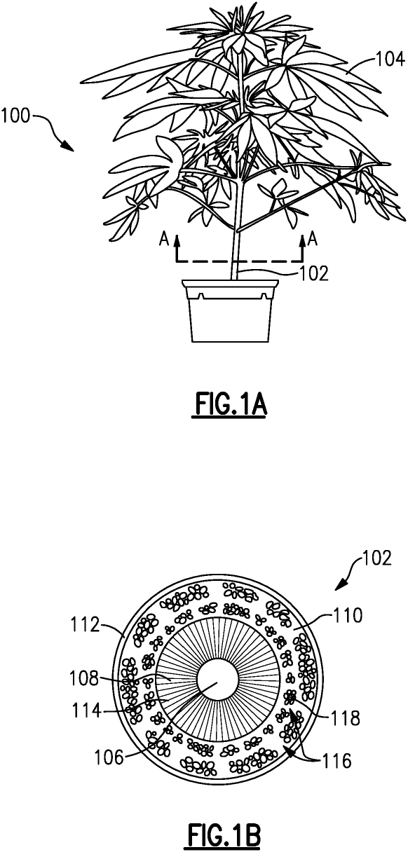

The cannabis plant also includes various forms of plant material. FIG. 1A schematically show a cannabis plant 100. The cannabis plant generally includes a stem 102 and leaves 104. FIG. 1B schematically shows a detail view of the stem 102 through the line A-A shown in FIG. 1A. The stem 102 includes a hollow inner core 106, a fibrous core 108, a bundle core 110 surrounding the fibrous core 108, and an outer bark or skin 112. The fibrous core 108 includes long fibers 114 called “hurd” fibers. The fibrous core 108 includes bundles 116 of a different, shorter fibers called “bast” fibers 118 arranged about the fibrous core 108.

The leaves 104 of the cannabis plant 100 are often processed for extraction of desirable chemicals such as those described above, leaving the stem 102 or other non-leaf parts of the cannabis plant 100 as a byproduct. The byproducts still contain the active chemicals and other compounds discussed above, but can be valued at a reduced cost as compared to the leaves 104, for example.

Active chemicals from cannabis can be used to treat or improve various disease states or other ailments. To that end, cannabis can be incorporated into a variety of medical apparatuses. The apparatuses can include implants, which are used in a wide variety of medical fields. Non-limiting examples include orthopedic devices (e.g., screws, plates, total joint replacements, etc.), cardiovascular devices (pacemakers, implantable defibrillators, stents, catheters, etc.), contraceptives, cosmetic implants, pain management devices, and others. These implants are known to cause inflammation at the implant site due to the body’s natural response to foreign objects. Also, implants can raise bacterial and fungal infection concerns.

Other example apparatuses can be placed on or near the body, such as bandages, patches, or the like. Tools, such as trocars, are also contemplated. These apparatuses can also trigger an inflammation or infection response, and thus would benefit from the properties of cannabis discussed herein.

The present disclosure relates to a material for a medical apparatus that contains cannabis and medical apparatuses comprising that material, which intrinsically exhibit at least one medicinally useful property due to the cannabis. The material and medical apparatuses of the present disclosure intrinsically have the benefit of the chemicals found in cannabis such as anti-inflammatory properties, without the inclusion of any other drugs.

Certain medical apparatuses are drug-eluting, e.g., they are loaded with a drug that leaves the apparatus and enters the body to have a medicinal effect. However, elution rates and effectiveness can be difficult to control and predict. Accordingly, because the presently disclosed apparatus intrinsically have desirable medical properties, the need for drug elution is eliminated or reduced.

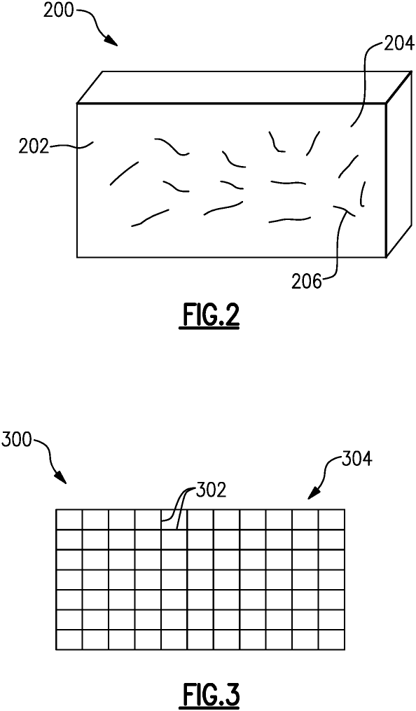

FIG. 2 shows an example medical apparatus 200 comprising a composite material 202. The composite material includes a matrix material 204 such as a polymeric matrix material 204. The matrix material 204 can be any material that is suitable for a medical apparatus, such as a polymeric material or a ceramic material. Non-limiting examples of suitable polymers include polyvinyl chloride (PVC), polypropylene (PP), polyethylene (PE), polystyrene (PS) as well as nylon, polyethylene terephthalate (PET), polyimide (PA), polycarbonate (PC), acrylonitrile butadiene (ABS), polyetheretherketone (PEEK), polyurethane (PU), and combinations thereof. A polymeric matrix material 204 can be tailored to have the desired mechanical properties for the resulting medical apparatus as would be known in the art. For instance, polymeric matrix material 204 for a stent may be somewhat flexible, whereas polymeric material for a knee implant may be rigid.

The composite material 202 also includes a cannabis phase 206. The cannabis phase 206 includes at least some byproduct material from cannabis plants 100, as discussed above. In some examples, the cannabis phase 206 can also include extracts from the cannabis plant 100 and/or leaves 102 of the cannabis plant 100. For instance, the extracts can be encapsulated to form cannabis-extract containing particles, and the particles could be incorporated into the matrix material 204. As noted above, the byproduct or other material from the cannabis plants 100 include active chemicals such as cannabinoids. The cannabis phase 206 this imparts intrinsic anti-inflammatory, anti-bacterial, anti-fungal, and/or other desirable properties to the medical apparatus 200.

The cannabis phase 206 is, in one example, fibers 114/118 as discussed above. The fibers could be hurd fibers 114, bast fibers 118, or a mixture of the two. In some examples, the fibers 114/118 may be treated or washed by any known method prior to inclusion in the material. Furthermore, the fibers may be ground or cut. The fibers 114/118 can be incorporated into the matrix 204 to form the composite material 202 by any known method/mechanism. For example, the fibers 114/118 can be arranged in a “preform” and then impregnated with the matrix material 204.

In one example, the cannabis phase 206 acts as a reinforcement phase in the composite material 202. That is, the cannabis phase 206 can act as a structural component of the medical apparatus 200. Where the cannabis phase 206 is fibers 114/118, as discussed above, fibers 114/118 can be woven, non-woven, knitted, braided, etc. The fibers 114/118 can be arranged in a particular manner to provide directional reinforcement, e.g., the fibers 114/118 can be arranged unidirectionally, in one example.

Other cannabis phases 206, such as particles, are also contemplated. For instance, byproduct material from the cannabis plant 100 as discussed above can be cut into small pieces or ground and incorporated into the matrix 204 during a molding process appropriate for that material (e.g., injection molding, compression molding, etc.).

Another example medical apparatus 300 is shown in FIG. 3. In this example, the medical apparatus 300 is comprised primarily of fibers 302 that are woven, braided, knitted, or otherwise formed into a fabric 304. The fabric 304 can be used as a bandage or gauze, for instance. The fibers 302 can be the cannabis byproduct fibers 114/118 discussed above. The fibers 302 can also include other types of fibers suitable for medical uses that are blended/combined with the cannabis fibers 114/118. The medical apparatus 300 can also be infused with extractions or other parts of the cannabis plant, as with medical apparatus 200 discussed above.

The preceding description is exemplary rather than limiting in nature. Variations and modifications to the disclosed examples may become apparent to those skilled in the art that do not necessarily depart from this disclosure. The scope of legal protection given to this disclosure can only be determined by studying the following claims.

What is claimed is:

1. A composite material for a medical apparatus, comprising:
a matrix material; and
a cannabis phase disposed in the matrix material, wherein the cannabis phase includes at least one active chemical.

2. The composite material of claim 1, wherein the cannabis phase includes at least one of terpenes and flavonoids.

3. The composite material of claim 1, wherein the cannabis phase includes fibers from a cannabis plant.

4. The composite material of claim 3, wherein the fibers are bast fibers, hurd fibers, or combinations thereof.

5. The composite material of claim 1, wherein the matrix is a polymeric material.

6. The composite material of claim 1, wherein the matrix is a ceramic material.

7. The composite material of claim 1, wherein the cannabis phase is ground or cut fibers of a cannabis plant.

8. The composite material of claim 1, wherein the cannabis phase includes at least one cannabis extract.

9. The composite material of claim 1, wherein the composite is formed into a medical apparatus configured to be implanted into a patient.

10. The composite material of claim 1, wherein the composite is formed into a tool configured to be used during a medical procedure.

11. The composite of claim 1, wherein the cannabis phase includes material from Cannabis sativa L .

12. A medical apparatus, comprising:
a cannabis phase, the cannabis phase including at least one of hurd fibers and bast fibers of a cannabis plant, wherein the medical apparatus intrinsically has at least one of anti-inflammatory, anti-bacterial, anti-fungal, antiemetic, orexigenic, and muscle relaxant properties due to the cannabis phase.

13. The medical apparatus of claim 9, wherein the medical apparatus is configured to be implanted in a patient.

14. The medical apparatus of claim 9, wherein the medical apparatus is a tool configured to be used in a medical procedure.

15. The medical apparatus of claim 9, wherein the medical apparatus is a fabric.

16. The composite of claim 1, wherein the cannabis phase includes material from Cannabis sativa L .

17. A method of making a medical apparatus, comprising:
forming fibers from a cannabis plant into a medical apparatus, whereby the medical apparatus intrinsically has at least one of anti-inflammatory, anti-bacterial, anti-fungal, antiemetic, orexigenic, and muscle relaxant properties due to the fibers.

18. The method of claim 17, further comprising disposing the fibers into a matrix phase.

19. The method of claim 17, further comprising weaving, knitting, or braiding the fibers.

20. The method of claim 17, wherein the medical apparatus is implantable into a patient.
